(19) United States
(12) Patent Application Publication
(10) Pub. No.: US 2008/0008740 A1
(43) Pub. Date: Jan. 10, 2008

(54) Pharmaceutical Composition for Rectal, or Vaginal Application, Method of Manufacturing and Use as Medicament Thereof

(76) Inventors: Ales Franc, Brno (CZ); Petr Sova, Hradec Kralove (CZ)

Correspondence Address: NOTARO AND MICHALOS, 100 DUTCH HILL ROAD, SUITE 110, ORANGEBURG, NY, 10962-2100, US

(21) Appl. No.: 11/574,775

(22) PCT Filed: Sep. 7, 2005
(86) PCT No.: PCT/CZ05/00068

§ 371 Date: Apr. 20, 2007

(30) Foreign Application Priority Data
Sep. 8, 2004 (CZ) PV 2004-945

(51) Int. Cl. A61K 9/02; A61K 31/282; A61K 9/14; A61P 43/00

(52) U.S. Cl. 424/430; 424/436; 424/499; 514/492

(57) ABSTRACT

A pharmaceutical composition for rectal or vaginal application, characterized in that it contains as active substance the platinum complex of general formula 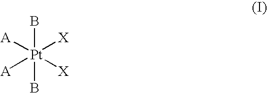 wherein
A each independently is an —NH₃ group or an amino group containing 1 to 18 carbon atoms, B each independently is a halogen atom, a hydroxy group or a —O—C(O)—R group wherein R each independently is hydrogen atom or an alkyl, alkenyl, aryl, aralkyl, alkylamino or alkoxy group containing 1 to 10 carbon atoms or functional derivatives of these groups, and X each independently is a halogen atom or a monocarboxylate group containing 1 to 20 carbon atoms, or X together form a dicarboxylate group containing 2 to 20 carbon atoms, and a hydrophilic gel-forming base, comprising gelatin, water and glycerol.

### FIELD OF INVENTION

This invention relates to a pharmaceutical composition for rectal or vaginal application, method of manufacturing and use as medicament thereof.

### BACKGROUND OF THE INVENTION

Platinum cytostatics, such as e.g. cisplatinum, carboplatinum or oxaliplatinum, are being used for many years in the treatment of solid tumors. However, these platinum cytostatics cannot be effectively applied orally because they are only sparingly soluble in water, unstable in the acidic medium of the stomach, and they are absorbed by the organism only with difficulty. Since in comparison with the alternative parenteral administration the oral application is much bearable and comfortable for the patient, there has been a need in the art for cytostatically effective platinum complexes whose solubility in water and absorbability by the organism would enable their oral application. Such orally applicable complexes of tetravalent platinum have been described in EP 0 328 274 and EP 0 423 707. Stability of these substances in an acidic medium, together with their physicochemical properties are favorable factors for their absorption by mucosa of the gastrointestinal tract. Nevertheless, in the form of particles, these complexes of tetravalent platinum have a high electrostatic charge that hampers their continuous processing to give a solid dosage form such as e.g. tablets. This disadvantage has been overcome by obtaining the mentioned platinum complexes in the form of inclusion complexes with cyclodextrins, their conversion into organic solution and subsequent lyophilization as described in WO 99/61451, or by processing using the wet granulation method as described in patent applications CZ 2003-915 and CZ 2004-235. According to the above patent applications, it is possible to use up to 350 mg of the active substance in a dose unit and to achieve enterosolvent and controlled release of the active pharmaceutical composition in the form of an oral solid dosage form.

Another possible application of platinum cytostatics is the rectal application, in which the resorbed drug avoids the decomposing aggressive medium of the gastrointestinal tract and after absorption by the rectal mucosa it passes via the portal system directly into the systemic blood circulation. The possibility of rectal application of a tetravalent platinum complex has been described in U.S. Pat. No. 6,033,683. Another possibility of application of platinum cytostatics is based on their administration in the form of vaginal globules in diseases of urogenital tract in women. The platinum cytostatic is released in the area of vagina and urethra and this local application enables the active substance to act directly at the tumor site.

Recently, there is an endeavor to apply oral complexes of tetravalent platinum of the type described in EP 0 328 274 and EP 0 423 707 also per rectum or per vagina in order to achieve a more potent effect of the mentioned complexes directly at the site of the tumor. Thus, the objective of the invention is to prepare a stable pharmaceutical composition, containing the mentioned tetravalent platinum complexes, that could be suitable for rectal and vaginal application.

This objective has been achieved by the pharmaceutical composition and the method of preparation thereof according to this invention.

### SUMMARY OF THE INVENTION

The invention relates to a pharmaceutical composition for rectal or vaginal application, characterized in that it contains as active substance a platinum complex of general formula I 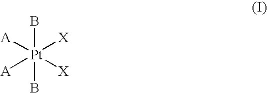
wherein

A each independently is an —NH₃ group or an amino group containing 1 to 18 carbon atoms,

B each independently is a halogen atom, a hydroxy group or a COOR group wherein R each independently is hydrogen atom or an alkyl, alkenyl, aryl, aralkyl, alkylamino or alkoxy group containing 1 to 10 carbon atoms or functional derivatives of these groups, and

X each independently is a halogen atom or a monocarboxylate group containing 1 to 20 carbon atoms, or X together form a dicarboxylate group containing 2 to 20 carbon atoms, and a hydrophilic gel-forming base, comprising gelatin, water and glycerol.

The pharmaceutical composition according to this invention advantageously contains at least one monosaccharide and/or disaccharide and/or polysaccharide.

The active substance in the pharmaceutical composition advantageously has such particle size distribution that 100% of the particles of the active substance are smaller than or equal to 100 micrometers, advantageously 80% of the particles of the active substance are smaller than or equal to 50 micrometers.

The pharmaceutical composition according to the invention advantageously is in the form of a unit dose containing 5 to 500 mg of the active substance, preferably 20 to 200 mg of the active substance.

The pharmaceutical composition according to the invention advantageously is in the form of a rectal suppository or a vaginal globule.

The invention also relates to a method of manufacturing the mentioned pharmaceutical composition, characterized in that the active substance is admixed with a thermally liquefied hydrophilic gel-forming base comprising gelatin, water and glycerol, whereupon the obtained mixture is shaped.

In the method according to the invention, the active substance is advantageously admixed with a thermally liquefied hydrophilic gel-forming base, in a mixture with at least one monosaccharide and/or disaccharide and/or polysaccharide with which the active substance had been pre-ground to the desired particle size.

According to the invention, the content of at least one monosaccharide and/or disaccharide and/or polysaccharide in the mixture with the active substance, intended for the pre-grinding, advantageously amounts to at least 20% by weight, preferably up to 50% by weight, based on the weight of the active substance.

According to the invention, the active substance is advantageously ground in a mixture with at least one monosaccharide and/or disaccharide and/or polysaccharide to achieve such particle size distribution that 100% of the particles of the active substance are smaller than or equal to 100 micrometers, 80% of the particles of the active substance being advantageously smaller than or equal to 50 micrometers.

According to the invention, the admixing of the active substance with the thermally liquefied hydrophilic gel-forming base is advantageously performed in a mixer in which the inner surface, coming into contact with the processed mixture, is a inert material, advantageously made of glass or ceramic, or it is teflon-coated or otherwise non-metallically modified.

According to the invention, the mixture obtained by admixing the active substance with the thermally liquefied hydrophilic gel-forming base is advantageously shaped by casting into rectal suppository or vaginal globule moulds, and then the cast mixture is left to cool down or cooled.

According to the invention, the mixture obtained by admixing the active substance with the molten hydrophilic gel-forming base, is advantageously shaped by casting into rectal suppository or vaginal globule moulds and subsequent leaving to cool down, or cooling, to give rectal suppositories or vaginal globules weighing 0.5 to 6 g.

The present invention also relates to the above defined pharmaceutical composition, or a composition prepared by the above-defined method, for use as a medicament for treatment of tumor diseases.

The pharmaceutical composition according to the invention comprises the mentioned platinum complex of general formula I, evenly suspended in a hydrophilic gel-forming base. Although the platinum complex of general formula I generally is incompatible with currently used pharmaceutically acceptable excipients, it has been surprisingly found that it is well compatible with a combination of components forming the hydrophilic gel-forming base according to the invention. Moreover, this base does not attack, and thus does not decompose, the mentioned platinum complex and enables its effective absorption by the rectal and vaginal mucosa. In grinding of the active substance to the desired particle size, it is possible to perform the grinding in the presence of at least one monosaccharide and/or disaccharide and/or polysaccharide, particularly in the presence of lactose, this compound serving as an auxiliary in the presence of which no substantial decomposition of the active substance takes place. This auxiliary also increases the viscosity of the mixture for preparation of rectal suppository or vaginal globule material, preventing thus sedimentation of the active substance during the preparation, solidification or gel formation. When using this excipient, a mixture of the active substance and the excipient may thus be added already into a solution of the hydrophilic gel-forming base of low viscosity. Dissolution of the excipient in the mentioned solution increases viscosity of the solution, thus inhibiting sedimentation of the active substance. When using the active substance alone, it is necessary to inhibit its sedimentation by adding the active substance to the hydrophilic gel-forming base only after cooling the base to a temperature ranging from about 40° C. to about 50° C. when the base exists already in the gel form.

In the following part the invention will be explained in more detail using individual examples of its execution which however are only illustrative and do not limit its scope. Within the framework of these examples, the platinum complex of general formula I employed is af-bis(acetato)-b-(1-adamantylamine)-c-ammine-de-dichloroplatinum(IV) complex of the following structural formula: 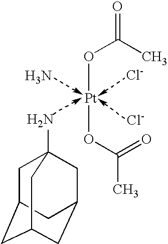
This complex of summary formula C₁₄H₂₆Cl₂N₂O₄Pt and of molecular weight 552.35 contains (acetato)-(1-adamantylamine)-amine-trichloroplatinum(IV) complex of structural formula Pt(ac)(am)(NH₃)Cl₃ as principal detectable impurity. The specific platinum(IV) complex employed, together with its antitumor effects, is described in the patent document PCT/CZ99/00015 where it is denoted as LA 12. It is sparingly soluble in water, its solubility being 0.03 g/100 ml, has a low bulk density amounting to 0.21 g/ml and a low tap density amounting to 0.42 g/ml, and an extremely high electrostatic charge.

### EXAMPLES

### Example 1

### Preparation of Rectal Suppositories

70 parts by weight of glycerol and 10 parts by weight of water are mixed together and the resulting mixture is heated to 70° C., whereupon 20 parts by weight of gelatin is added and the mixture obtained is slowly stirred for 45 minutes to dissolve the gelatin. Then the mixture is cooled to a temperature ranging from about 40° C. to about 50° C. to obtain a gel into which 20 parts by weight of the platinum(IV) complex LA 12 is added under slow stirring. The stirring is continued until a homogeneous gel suspension is obtained which is then cast into moulds for shaping rectal suppositories, the moulds being pre-hydrophobized by spreading with paraffin oil. The mould content is left to cool to room temperature and the rectal suppositories are taken out and adjusted in blisters.

### Example 2

### Preparation of Vaginal Globules

70 parts by weight of glycerol and 10 parts by weight of water are mixed together and the resulting mixture is heated to 70° C., whereupon 20 parts by weight of gelatin is added and the mixture obtained is slowly stirred for 45 minutes to dissolve the gelatin. To this mixture is added, with slow stirring, a mixture of 40 parts by weight of lactose and 20 parts by weight of platinum(IV) complex LA 12, which mixture had been pre-ground in a ball mill in order to satisfy the condition that 80% of particles of the platinum complex LA 12 are smaller than or equal to 50 micrometers. The stirring at the temperature mentioned is continued until the lactose is dissolved. Then, this mixture is cooled to a temperature ranging from about 40° C. to about 50° C. to obtain a homogeneous gel suspension which is then cast into moulds for shaping vaginal globules, the moulds being pre-hydrophobized by spreading with paraffin oil. The mould content is left to cool to room temperature and the vaginal globules are taken out and adjusted in blisters.

1: A pharmaceutical composition for rectal or vaginal application, characterized in that it contains as active substance the platinum complex of general formula 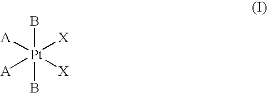
wherein
A each independently is an —NH₃ group or an amino group containing 1 to 18 carbon atoms,
B each independently is a halogen atom, a hydroxy group or a —O—C(O)—R group wherein R each independently is hydrogen atom or an alkyl, alkenyl, aryl, aralkyl alkylamino or alkoxy group containing 1 to 10 carbon atoms or functional derivatives of these groups, and
X each independently is a halogen atom or a monocarboxylate group containing 1 to 20 carbon atoms, or X together form a dicarboxylate group containing 2 to 20 carbon atoms, and a hydrophilic gel-forming base, comprising gelatin, water and glycerol.

2: The pharmaceutical composition according to claim 1, characterized in that it comprises at least one monosaccharide and/or disaccharide and/or polysaccharide.

3: A pharmaceutical composition according to claim 1, characterized in that the active substance has such particle size distribution that 100% of particles of the active substance are of size smaller than or equal to 100 micrometers, preferably 80% of particles of the active substance are of size smaller than or equal to 50 micrometers.

4: A pharmaceutical composition according to claim 1, characterized in that it is in the form of a unit dose comprising 5 to 500 mg of the active substance, advantageously 20 to 200 mg of the active substance.

5: A pharmaceutical composition according to claim 1, characterized in that it is in the form of a rectal suppository or a vaginal globule.

6: A method of manufacturing pharmaceutical composition according to claim 1, characterized in that the active substance is admixed with a thermally liquefied hydrophilic gel-forming base comprising gelatin, water and glycerol, whereupon the mixture obtained is shaped

7: The method according to claim 6, characterized in that a mixture of the active substance with at least one monosaccharide and/or disaccharide and/or polysaccharide, with which the active substance had been pre-ground to obtain the desired particle size, is admixed with thermally liquefied hydrophilic gel-forming base.

8: The method according to claim 7, characterized in that the content of at least one monosaccharide and/or disaccharide and/or polysaccharide in the mixture with the active substance intended for the pre-grinding equals to or is higher than 20% by weight, preferably up to 50% by weight, based on the weight of the active substance.

9: A method according to claim 7, characterized in that the active substance is ground in a mixture with at lest one monosaccharide and/or disaccharide and/or polysaccharide to obtain such particle size distribution that 100% of the particles of the active substance are of size smaller than or equal to 100 micrometers, preferably 80% of the particles of the active substance are of size smaller than or equal to 50 micrometers.

10: A method according to claim 6, characterized in that admixing the active substance with the thermally liquefied hydrophilic gel-forming base is performed in a mixer in which the inner surface, coming into contact with the processed mixture, is made of a inert material, advantageously of glass or ceramic, or of a teflon-coated, or otherwise non-metallically modified material

11: A method according to claim 6, characterized in that the mixture, obtained by admixing the active substance with the thermally liquefied hydrophilic gel-forming base, is shaped by casting into moulds for rectal suppositories or vaginal globules, and subsequent leaving to cool down or cooling of the cast mixture.

12: The method according to claim 11, characterized in that the mixture, obtained by admixing the active substance with the thermally liquefied hydrophilic gel-forming base, is shaped by casting into moulds for rectal suppositories or vaginal globules, and after leaving to cool down or cooling of the cast mixture, affords rectal suppositories or vaginal globules weighing 0.5 g to 6 g.

13. (canceled)
